(19) United States
(12) Patent Application Publication
(10) Pub. No.: US 2025/0305009 A1
(43) Pub. Date: Oct. 2, 2025

(54) POLYHYDROXYALKANOATE PRODUCTION METHOD

(71) Applicant: KANEKA CORPORATION, Osaka-shi (JP)

(72) Inventors: Yoshihiro MOURI, Takasago-shi (JP); Hisashi ARIKAWA, Takasago-shi (JP); Shunsuke SATO, Takasago-shi (JP)

(73) Assignee: KANEKA CORPORATION, Osaka-shi (JP)

(21) Appl. No.: 19/238,782

(22) Filed: Jun. 16, 2025

Related U.S. Application Data
(63) Continuation of application No. PCT/JP2023/044038, filed on Dec. 8, 2023.

(30) Foreign Application Priority Data
Dec. 20, 2022 (JP) 2022-203396

(51) Int. Cl. C12P 7/625; C12N 1/20; C12R 1/01

(52) U.S. Cl. CPC C12P 7/625; C12N 1/205; C12R 2001/01

(57) ABSTRACT

A polyhydroxyalkanoate production method includes culturing a polyhydroxyalkanoate-producing microorganism in a culture medium to obtain microbial cells accumulating a polyhydroxyalkanoate. At start of the culturing of the polyhydroxyalkanoate-producing microorganism, the culture medium contains a sulfur source at a sulfur concentration of 0.0001 to 13 mM. The polyhydroxyalkanoate production method includes the step of adding a carbon source and a sulfur source to the culture medium during the culturing of the polyhydroxyalkanoate-producing microorganism, and an average ratio (C/S ratio) of a carbon weight (C) in the carbon source added per hour to a sulfur weight (S) in the sulfur source added per hour, as calculated for a period in which the sulfur source is added, is in a range of 500 to 10,000.

### TECHNICAL FIELD

The present invention relates to a polyhydroxyalkanoate production method using microbial culture.

### BACKGROUND ART

Sustainability-related issues including SDGs are increasingly attracting global attention, and there is a growing awareness of environmental problems such as the problem of marine microplastics. Against this background, substitution of biodegradable materials for existing non-biodegradable plastics derived from petroleum is being promoted, especially in industries such as packaging and food services, biomedicine, and agriculture. Examples of biodegradable materials the industrial production of which has been actively pursued in recent years include polylactic acid (PLA) and polyhydroxyalkanoates (PHAs). In particular, PHAs exhibit high biodegradability in a wide variety of environments and are a rare class of biodegradable materials that are biodegradable even in seawater. For this reason, PHAs are increasingly expected as a solution to the problem of marine microplastics and other environmental problems.

PHAs are naturally occurring thermoplastic polyesters produced and accumulated as energy storage substances in the cells of many kinds of microorganisms. In general, a PHA is industrially produced by supplying nutrient sources to a PHA-accumulating microorganism and culturing the microorganism.

Commonly known culture methods used in microbial material production include batch culture (a technique in which necessary nutrient components are added to the culture medium at the start of the culture), continuous culture (a technique in which the concentration of a certain nutrient component in the culture fluid is kept constant by addition of the nutrient component and discharge of the culture fluid), and fed-batch culture (a technique in which a certain nutrient component is added without discharge of the culture fluid).

The batch culture is a culture method suitable for small-scale production and is most frequently used at the laboratory level. With the use of this method, obtaining the intended product at a higher concentration requires a larger amount of necessary nutrient components at the start of the culture. However, the nutrient components include a component that exhibits cytotoxicity when present at a high concentration. Thus, the batch culture is rarely employed in microbial culture-based material production performed at the industrial level.

In terms of reducing the carbon source concentration at the start of the culture, a technique such as the fed-batch culture or continuous culture is desired in which a carbon source is added in the course of the culture. Thus, many techniques in which a carbon source is added in the course of the culture have been proposed (see Patent Literatures 1 and 2, for example).

A well-known feature of PHA-accumulating microorganisms is that in an environment containing an abundance of carbon source, the microorganisms undergo a metabolic change induced by phosphorus source depletion and/or nitrogen source depletion and thus accumulate PHAs. For this reason, when a PHA-accumulating microorganism is cultured, the phosphorus source concentration and/or nitrogen source concentration at the start of the culture is also limited to a certain extent. Meanwhile, it has been reported that phosphorus source addition following phosphorus source depletion or nitrogen source addition following nitrogen source depletion is effective to improve the PHA productivity (see Patent Literatures 2 and 3, for example).

### CITATION LIST

### Patent Literature

PTL 1: Japanese Laid-Open Patent Application Publication No. 2015-006181
PTL 2: Japanese Laid-Open Patent Application Publication No. 2005-080529
PTL 3: Japanese Laid-Open Patent Application Publication No. 2013-9628

### SUMMARY OF INVENTION

### Technical Problem

As described above, it is known that in PHA production by microbial culture, there are restrictions on the concentrations of carbon, phosphorus, and nitrogen sources at the start of the culture. However, few findings are known about sulfur sources.

A sulfur source is used also in PHA production culture. In general, the sulfur concentration at the start of the culture is often set in the range of about 10 to about 40 mM. However, as far as the present inventors know, there are no findings about the impact that the sulfur concentration at the start of and during the culture has on the PHA productivity.

In view of the above circumstances, the present invention aims to achieve enhanced PHA productivity in producing a PHA by culture of a PHA-producing microorganism.

### Solution to Problem

The present inventors have found that in the case where a PHA-producing microorganism is cultured along with addition of a carbon source during the culture, the PHA production is inhibited if the sulfur concentration of a sulfur source contained in the culture medium at the start of the culture is higher than 13 mM. The present inventors have further found that the PHA productivity can be enhanced by setting the sulfur concentration of a sulfur source contained in the culture medium at the start of the culture to 13 mM or lower and adding a given amount of sulfur source in the course of the culture. These findings have led the inventors to the present invention.

Specifically, the present invention relates to a polyhydroxyalkanoate production method including culturing a polyhydroxyalkanoate-producing microorganism in a culture medium to obtain microbial cells accumulating a polyhydroxyalkanoate, wherein
at start of the culturing of the polyhydroxyalkanoate-producing microorganism, the culture medium contains a sulfur source at a sulfur concentration of 0.0001 to 13 mM,
the polyhydroxyalkanoate production method includes the step of adding a carbon source and a sulfur source to the culture medium during the culturing of the polyhydroxyalkanoate-producing microorganism, and
an average ratio (C/S ratio) of a carbon weight (C) in the carbon source added per hour to a sulfur weight (S) in the sulfur source added per hour, as calculated for a period in which the sulfur source is added, is in a range of 500 to 10,000.

### Advantageous Effects of Invention

The present invention allows for achieving enhanced PHA productivity in producing a PHA by culture of a PHA-producing microorganism.

The present invention allows for achieving enhanced PHA productivity by fed-batch culture or continuous culture of a PHA-producing microorganism.

According to a preferred aspect of the present invention, the total amount of the sulfur source used can be reduced. This makes it possible to efficiently enhance the PHA productivity using a fermenter having a limited volume. Furthermore, the sulfur content in wastewater discharged after the microbial culture is reduced, and this can reduce the cost involved in wastewater treatment concerning sulfur.

### DESCRIPTION OF EMBODIMENTS

Hereinafter, an embodiment of the present invention will be described in detail.

The embodiment of the present invention relates to a polyhydroxyalkanoate production method including culturing a polyhydroxyalkanoate-producing microorganism in a culture medium to obtain microbial cells accumulating a polyhydroxyalkanoate.

### (Polyhydroxyalkanoate)

The polyhydroxyalkanoate (PHA) is not limited to a particular type and may be any PHA that can be produced by a microorganism. The PHA may be a homopolymer consisting of one hydroxyalkanoate or a copolymer consisting of two or more hydroxyalkanoates. Specific examples of the PHA include: a homopolymer of one monomer selected from 3-hydroxyalkanoates having 4 to 16 carbon atoms; a copolymer of one monomer selected from 3-hydroxyalkanoates having 4 to 16 carbon atoms and another hydroxyalkanoate (such as a 2-hydroxyalkanoate, 4-hydroxyalkanoate, 5-hydroxyalkanoate, or 6-hydroxyalkanoate having 4 to 16 carbon atoms); and a copolymer of two or more monomers selected from 3-hydroxyalkanoates having 4 to 16 carbon atoms.

Among others, a homopolymer or copolymer containing 3-hydroxybutyrate as monomer units is preferred. Examples of such a polymer include, but are not limited to, P(3HB) which is a homopolymer of 3-hydroxybutyrate (abbreviated as 3HB), P(3HB-co-3HV) which is a copolymer of 3HB and 3-hydroxyvalerate (abbreviated as 3HV), P(3HB-co-3HH) (abbreviated as PHBH) which is a copolymer of 3HB and 3-hydroxyhexanoate (abbreviated as 3HH), P(3HB-co-4HB) which is a copolymer of 3HB and 4-hydroxybutyrate (abbreviated as 4HB), and a PHA containing lactic acid (abbreviated as LA) as a constituent (an example of this PHA is P(LA-co-3HB) which is a copolymer of 3HB and LA). Among these, PHBH is preferred since this polymer has a wide range of applications.

The type of the PHA to be produced can be chosen as appropriate according to the intended purpose and can be changed depending on factors such as the type of the PHA synthase gene possessed by or introduced into the microorganism used, the type of the metabolic gene involved in the PHA synthesis, and the culture conditions.

### (PHA-Producing Microorganism)

The PHA-producing microorganism may be any microorganism having the ability to produce a PHA. The microorganism may be a microorganism having a PHA synthase gene. The microorganism may be a wild strain that inherently has a PHA synthase gene, a mutant strain obtained by artificially mutating the wild strain, or a strain having an exogenous PHA synthase gene introduced by a genetic engineering technique.

The PHA-producing microorganism is not limited to a particular type and may be any microorganism having the PHA-producing ability. The PHA-producing microorganism may be a microorganism found in nature, a mutant, or a transformant. Specific examples include: bacteria of the genus Cupriavidus such as Cupriavidus necator; bacteria of the genus Alcaligenes such as Alcaligenes latus; bacteria of the genus Pseudomonas such as Pseudomonas putida, Pseudomonas fluorescens, Pseudomonas aeruginosa, Pseudomonas resinovorans, and Pseudomonas oleovorans; bacteria of the genus Bacillus such as Bacillus megaterium; bacteria of the genus Azotobacter; bacteria of the genus Nocardia; bacteria of the genus Aeromonas such as Aeromonas caviae and Aeromonas hydrophila; bacteria of the genus Ralstonia; bacteria of the genus Wautersia; and bacteria of the genus Comamonas (Microbiological Reviews, pp. 450-472, 1990). Biological cells can also be used which have been artificially modified by introducing a PHA synthase gene or the like through a genetic engineering technique and which have thus become able to produce a PHA. For example, the following organisms can be used: gram-negative bacteria such as bacteria of the genus Escherichia; gram-positive bacteria such as bacteria of the genus Bacillus; yeasts such as yeasts of the genus Saccharomyces, Yarrowia, or Candida; and cells of higher organisms such as plants. Bacteria are preferred since they can accumulate a large amount of PHA. Bacteria of the genus Cupriavidus are more preferred and Cupriavidus necator is particularly preferred.

### (PHA Synthase Gene)

The PHA synthase gene introduced through genetic transformation is not limited to a particular type. Examples of the PHA synthase gene include: PHA synthase genes derived from Aeromonas caviae, Aeromonas hydrophila, Pseudomonas SP 61-3, and Cupriavidus necator; and altered genes resulting from alteration of these PHA synthase genes. The term “altered gene” refers to a base sequence that encodes a PHA synthase having an amino acid sequence in which one or more amino acid residues have been deleted, added, inserted, or replaced.

### (Culture)

The culture of a polyhydroxyalkanoate-producing microorganism in the present embodiment refers to “main culture” performed at the final stage to allow the polyhydroxyalkanoate-producing microorganism to accumulate a polyhydroxyalkanoate at a high concentration. “Preculture” and “seed culture” prior to the “main culture” are not included in the “culture” in the present embodiment. Thus, in the “preculture” and “seed culture”, the sulfur concentration is not limited to a particular range, and there is no particular limitation on whether or not a sulfur source is added during the culture.

### (Culture Media)

The culture media used in the “preculture”, “seed culture”, and “main culture” may be any liquid culture media containing nutrient sources conducive to the growth and proliferation of the polyhydroxyalkanoate-producing microorganism to be cultured. It is preferable to mix the PHA-producing microorganism with a liquid containing a carbon source, a nitrogen source, a phosphorus source, a sulfur source, an inorganic salt, and another organic nutrient source, and stir or shake the mixture to disperse the PHA-producing microorganism.

Examples of the nitrogen source include ammonia and ammonium salts such as ammonium chloride, ammonium sulfate, and ammonium phosphate and further include nitric acid, nitrate salts, nitrite salts, peptone, meat extract, and yeast extract. Examples of the phosphorus source include phosphate salts such as potassium dihydrogen phosphate, disodium hydrogen phosphate, magnesium phosphate, and ammonium phosphate and further include inorganic phosphoric acid, peptone, meat extract, and yeast extract. Examples of the inorganic salt include chlorides, phosphates, nitrates, nitrites, sulfates, and sulfites of magnesium, sodium, potassium, and trace metal elements (such as iron, cobalt, nickel, and copper). Examples of the other organic nutrient source include: amino acids such as glycine, alanine, serine, threonine, and proline; and vitamins such as vitamin Bi, vitamin B12, and vitamin C. For the culture media used in the “preculture” and “seed culture”, any nutrient sources conducive to the growth and proliferation of the selected polyhydroxyalkanoate-producing microorganism can be freely selected. In the “main culture” in the present embodiment, a nutrient source containing sulfur element is preferably used in accordance with “Way of Using Sulfur Source” described later.

### (Carbon Source)

The “main culture” of the polyhydroxyalkanoate-producing microorganism in the present embodiment includes the step of adding a carbon source to the culture medium during the culture. The addition of the carbon source is not limited to a particular way of addition but preferably consecutive addition. That is, the “main culture” is preferably performed along with consecutive addition of the carbon source to the culture medium containing the PHA-producing microorganism. The term “consecutive addition” as used herein includes the way of addition in which the carbon source is continuously added without any interruption and the way of addition in which the carbon source is intermittently added a plurality of times at intervals.

Examples of the carbon source include: oils and fats containing glycerides and fatty acids; sugars such as glucose and fructose; and organic carbon sources such as peptone, meat extract, and yeast extract. Examples of the oils and fats that can be used include, but are not limited to, animal oils and fats, vegetable oils and fats, mixtures of animal and vegetable oils and fats, transesterified oils, and fractionated oils. Specific examples of the vegetable oils and fats include rapeseed oil, sunflower oil, soybean oil, olive oil, corn oil, palm oil, palm kernel oil, cottonseed oil, sesame oil, nut oil, Jatropha oil, and rice oil. Specific examples of the animal oils and fats include lard. One of the above-mentioned substances may be used alone, or a mixture of two or more thereof may be used.

The amount of the carbon source added during the consecutive addition is not limited to a particular value. The addition of the carbon source is preferably done so that the carbon source concentration in the culture medium will be kept within a given range.

### (Way of Using Sulfur Source)

In the “main culture” of the polyhydroxyalkanoate-producing microorganism in the present embodiment, the culture medium contains a sulfur source at a sulfur concentration of 0.0001 to 13 mM at the start of the culture. If the sulfur concentration in the culture medium is higher than 13 mM at the start of the culture, the sulfur source inhibits the PHA production, thus making it difficult to achieve good PHA productivity. The sulfur concentration may be 10 mM or lower, 8 mM or lower, or 6 mM or lower.

The sulfur concentration at the start of the culture is at least 0.0001 mM and may be 0.001 mM or higher, 0.01 mM or higher, 0.1 mM or higher, 0.5 mM or higher, 1 mM or higher, 3 mM or higher, and 5 mM or higher.

The “sulfur concentration in the culture medium at the start of the culture” is the molar amount (mol) of the sulfur element contained in the culture fluid per unit volume (L) of the culture fluid into which a seed culture fluid has been inoculated. The “sulfur concentration in the culture medium at the start of the culture” can be calculated from the composition of the culture medium in the case where the sulfur source contained in the culture medium is composed entirely of an inorganic salt. In the case where sulfur is brought in from the seed culture fluid, the amount of the brought-in sulfur is calculated based on the composition of the culture medium of the seed culture fluid and the amount of the inoculated seed culture fluid, and the calculated value is combined with the amount of sulfur calculated from the composition of the culture medium of the main culture fluid.

In the case where the sulfur source contained in the culture medium includes an organic nutrient source, the “sulfur concentration in the culture medium at the start of the culture” can be calculated by extracting part of the culture medium as prepared and measuring the sulfur element content in the collected culture medium by means of an elemental analyzer.

It is preferable to use an inorganic salt since in this case the sulfur concentration at the start of the culture can be easily adjusted.

The sulfur source used at the start of the culture is not limited to a particular substance and can be selected from: inorganic salts such as magnesium sulfate, potassium sulfate, sodium sulfate, ammonium sulfate, and sulfates and sulfites of trace metal elements (such as iron, cobalt, nickel, and copper); sulfuric acid; and organic nutrient sources such as peptone, meat extract, and yeast extract. One of these sulfur sources may be used alone, or two or more of the sulfur sources may be used in combination. Among others, sulfuric acid or an inorganic salt is preferred, and sulfuric acid or a sulfate salt is more preferred.

The “main culture” of the polyhydroxyalkanoate-producing microorganism in the present embodiment includes the step of adding a sulfur source to the culture medium during the culture. The sulfur source added during the culture is not limited to a particular substance and can be selected from: inorganic salts such as magnesium sulfate, potassium sulfate, sodium sulfate, ammonium sulfate, and sulfates and sulfites of trace metal elements (such as iron, cobalt, nickel, and copper); sulfuric acid; and organic nutrient sources such as peptone, meat extract, and yeast extract. One of these sulfur sources may be used alone, or two or more of the sulfur sources may be used in combination. In terms of the ease of adjustment of the amount of the sulfur source added, sulfuric acid or an inorganic salt is preferred and sulfuric acid or a sulfate salt is more preferred. The sulfur source may be added by itself. Alternatively, the sulfur source may be dissolved or dispersed in water, and the solution or dispersion may be added.

The addition of the sulfur source to the culture medium during the culture is not limited to a particular way of addition but preferably consecutive addition. That is, the “main culture” is preferably performed along with consecutive addition of the sulfur source to the culture medium containing the PHA-producing microorganism. The term “consecutive addition” as used herein includes the way of addition in which the sulfur source is continuously added without any interruption and the way of addition in which the sulfur source is intermittently added a plurality of times at intervals.

The consecutive addition of the sulfur source to the culture medium during the culture may be commenced upon the start of the culture. Preferably, the addition of the sulfur source is commenced after a certain time has elapsed from the start of the culture and the sulfur concentration in the culture medium has decreased. For example, the addition of the sulfur source may be commenced 1 hour or more, 3 hours or more, or 5 hours or more, after the start of the culture.

In the addition of the carbon source and the sulfur source to the culture medium during the culture, it is desired that the amounts of the carbon and sulfur sources added be controlled as follows. That is, the amounts of the carbon and sulfur sources added are controlled so that an average ratio (C/S ratio) of the carbon weight (C) in the carbon source added per hour to the sulfur weight (S) in the sulfur source added per hour will be in the range of 500 to 10,000. The average C/S ratio is determined by setting 1-hour periods as unit time periods, calculating the C/S ratio for each unit time period, and averaging the calculated C/S ratios. In other words, the amount of the sulfur source added during the culture is on average from 1/10,000 to 1/500 of the amount of the carbon source added during the culture and is considerably small compared to the amount of the carbon source.

The C/S ratio is calculated for a “period in which the sulfur source is added”.

The “period in which the sulfur source is added” refers to the period from the start of the addition of the sulfur source to the end of the addition of the sulfur source. The start point of the “period in which the sulfur source is added” is the time point at which the addition of the sulfur source is started. That is, the period from the start of the culture to the start of the addition of the sulfur source is excluded from the “period in which the sulfur source is added”.

Basically, the end point of the “period in which the sulfur source is added” is the time point at which the last addition of the sulfur source before the end of the culture is completed. However, it is conceivable that the last one of the unit time periods, into which the “period in which the sulfur source is added” is divided at intervals of 1 hour from the start point of the “period in which the sulfur source is added”, is less than 1 hour and the culture is continued after completion of the last addition of the sulfur source. In such a case, the time point at which 1 hour has elapsed from the start of the last unit time period is regarded as the end point of the “period in which the sulfur source is added”. In the case where the last unit time period is less than 1 hour and the culture is ended during the last unit time period, the time point at which the culture is ended is regarded as the end point of the “period in which the sulfur source is added”.

The “ratio (C/S ratio) of the carbon weight (C) in the carbon source added per hour to the sulfur weight (S) in the sulfur source added per hour” refers to an average C/S ratio calculated as follows: the “period in which the sulfur source is added” is divided into unit time periods at intervals of 1 hour from the start point of the “period in which the sulfur source is added”, the C/S ratio is calculated for each 1-hour unit time period, and the C/S ratios obtained for the unit time periods are averaged to determine the average C/S ratio. In the case where the last unit time period is less than 1 hour, the time period of less than 1 hour is also taken into account as a unit time period, and the value of the C/S ratio in this unit time period is used in the calculation of the average C/S ratio.

In the case where the carbon source is not added during a unit time period, the C/S ratio in this unit time period is zero. This C/S ratio (zero) is not taken into account in the calculation of the average C/S ratio. In the case where the amount of the sulfur source added during a unit time period is zero or considerably small compared to the amount of the carbon source added during the same unit time period, the effect of the addition of the sulfur source cannot be achieved in such a unit time period. Thus, when the C/S ratios calculated for the unit time periods include a C/S ratio of more than 20,000, such a high C/S ratio is not taken into account in the calculation of the average C/S ratio.

If the average C/S ratio is less than 500, the total amount of the sulfur source used is large, so that the effect of the addition of the sulfur source could plateau or that the sulfur source could inhibit the PHA production to make it difficult to achieve good PHA productivity. In addition, the increase in the total amount of the sulfur source used could increase the burden of wastewater treatment, thus resulting in increased cost. If the average C/S ratio is more than 10,000, the amount of the sulfur source added is small, so that it could be difficult to achieve good PHA productivity. In terms of achieving good PHA productivity while moderating the total amount of the sulfur source used, the average C/S ratio is preferably from 1,000 to 6,000 and more preferably from 1,000 to 4,000.

The culture method may be continuous culture or fed-batch culture.

The culture conditions may be set as per ordinary microbial culture, except for those concerning the above-described addition of the carbon source and the sulfur source. There are no particular limitations on the culture scale, the aeration/stirring conditions, and the pH during the culture. The culture temperature may be selected as appropriate for proliferation and PHA production of the microorganism to be grown. For example, the culture temperature is preferably from about 20 to about 40° C. The culture time may also be set as appropriate and is preferably from about 1 to about 7 days.

During the culture, the phosphorus source and/or the nitrogen source may be added at one time or consecutively as appropriate in addition to the carbon source and the sulfur source.

The amount of the PHA accumulated in the PHA-producing microorganism at the end of the culture is not limited to a particular range and may be chosen as appropriate. The amount of the accumulated PHA is preferably 80 wt % or more and more preferably 90 wt % or more.

### (Collection of PHA)

After the microorganism is cultured for a suitable time to allow the microorganism to accumulate a PHA in its cells, the PHA is collected from the microbial cells using a known method. The collection of the PHA is not limited to using a particular method and can be accomplished, for example, as follows. The microbial cells are separated from the culture fluid by means such as a centrifuge, and the separated microbial cells are washed with a liquid such as distilled water or methanol and then dried. The PHA is extracted from the dried microbial cells using an organic solvent such as chloroform. The cellular components are removed from the PHA-containing solution through a process such as filtration, and a poor solvent such as methanol or hexane is added to the filtrate to precipitate the PHA. The supernatant fluid is removed through filtration or centrifugation, and the PHA is dried and collected.

In another example, the microbial cells are separated from the culture fluid by means such as a centrifuge, and the separated microbial cells are washed with a liquid such as distilled water or methanol. Subsequently, the washed sample is mixed with a solution of sodium lauryl sulfate (SDS), and the mixture is subjected to ultrasonic disruption to break the cell membranes. The cellular components and the PHA are then separated by means such as a centrifuge, and the PHA is dried and collected.

### (PHA Productivity)

The PHA productivity can be evaluated by the amount (g/L) of the PHA contained per L of the culture fluid after the end of the culture. Specifically, the PHA is collected from a certain volume of the culture fluid by any PHA collection method as described above, the weight of the collected PHA is measured, the obtained PHA weight is divided by the volume of the culture fluid to calculate the PHA productivity. The method for PHA collection may be selected as appropriate. When the PHA productivity is compared between samples, the same PHA collection method is selected for all the samples.

In the present embodiment, where a sulfur source is added in a given amount during the culture, a PHA can be produced at higher PHA productivity than under culture conditions where no sulfur source is added during the culture.

In the following items, preferred aspects of the present disclosure are listed. The present invention is not limited to the following items.

### Item 1

A polyhydroxyalkanoate production method including culturing a polyhydroxyalkanoate-producing microorganism in a culture medium to obtain microbial cells accumulating a polyhydroxyalkanoate, wherein
at start of the culturing of the polyhydroxyalkanoate-producing microorganism, the culture medium contains a sulfur source at a sulfur concentration of 0.0001 to 13 mM,
the polyhydroxyalkanoate production method includes the step of adding a carbon source and a sulfur source to the culture medium during the culturing of the polyhydroxyalkanoate-producing microorganism, and
an average ratio (C/S ratio) of a carbon weight (C) in the carbon source added per hour to a sulfur weight (S) in the sulfur source added per hour, as calculated for a period in which the sulfur source is added, is in a range of 500 to 10,000.

### Item 2

The polyhydroxyalkanoate production method according to item 1, wherein the average C/S ratio is from 1,000 to 6,000.

### Item 3

The polyhydroxyalkanoate production method according to item 1 or 2, wherein the sulfur source includes at least one substance selected from the group consisting of sulfuric acid and a sulfate salt.

### Item 4

The polyhydroxyalkanoate production method according to any one of items 1 to 3, wherein the polyhydroxyalkanoate-producing microorganism belongs to the genus Cupriavidus.

### Item 5

The polyhydroxyalkanoate production method according to any one of claims 1 to 4, wherein the polyhydroxyalkanoate-producing microorganism is Cupriavidus necator.

### Item 6

The polyhydroxyalkanoate production method according to any one of claims 1 to 5, wherein the polyhydroxyalkanoate is a copolymer containing at least 3-hydroxybutyrate and 3-hydroxyhexanoate as monomer units.

### EXAMPLES

Hereinafter, the present invention will be described more specifically using examples. The present invention is not limited to the examples given below.

In all of Reference Examples, Comparative Examples, and Examples described below, KNK-005 was used as a PHA-producing microorganism.

The KNK-005 is a transformant prepared according to a method described in U.S. Pat. No. 7,384,766 and having an Aeromonas caviae-derived PHA synthase gene introduced on the chromosome of Cupriavidus necator H16.

### Reference Example 1

The KNK-005 was used to sequentially perform (1) preculture, (2) seed culture, and (3) main culture according to the procedures described below.

### (1) Preculture

First, 20 μl of glycerol stock of the KNK-005 was inoculated into 20 mL of a preculture medium and cultured at 30° C. for 18 hours.

The preculture medium was composed of 1 w/v % Meat-extract, 1 w/v % Bacto-Tryptone, 0.2 w/v % Yeast-extract, 0.9 w/v % Na₂HPO₄·12H₂O, and 0.15 w/v % KH₂PO₄ (pH=6.8).

### (2) Seed Culture

The preculture fluid obtained as above was inoculated at a concentration of 1.0 v/v % into a 3-L jar fermenter (MDL-8C manufactured by B.E. Marubishi Co., Ltd.) containing 1.8 L of a seed culture medium. The fermenter was operated at a culture temperature of 30° C., a stirring speed of 500 rpm, and an aeration of 1.8 L/min, and the seed culture was conducted for 24 hours during which the pH was controlled between 6.5 and 6.6. For the pH control, a 14% aqueous solution of ammonium hydroxide was used.

The seed culture medium was composed of 0.385 w/v % Na₂HPO₄·12H₂O, 0.067 w/v % KH₂PO₄, 0.15 w/v % (NH₄)₂SO₄, 0.1 w/v % MgSO₄·7H₂O, 0.155 w/v % NH₄Cl, 2.5 w/v % palm olein oil, and 0.5 v/v % trace metal salt solution (solution of 1.6 w/v % FeCl₃·6H₂O, 1 w/v % CaCl₂·2H₂O, 0.02 w/v % CoCl₂·6H₂O, 0.016 w/v % CuSO₄·5H₂O, and 0.012 w/v % NiCl₂·6H₂O in 0.1 N hydrochloric acid).

### (3) Main Culture

The seed culture fluid obtained as above was inoculated at a concentration of 5.0 v/v % into a 5-L jar fermenter (Bioneer-Neo manufactured by B.E. Marubishi Co., Ltd.) containing 1.8 L of a main culture medium. The fermenter was operated at a culture temperature of 30° C., a stirring speed of 500 rpm, and an aeration of 3.0 L/min, and the pH was controlled between 6.3 and 6.7. For the pH control, a 25% aqueous solution of ammonium hydroxide was used.

In the main culture, palm olein oil was intermittently added as a carbon source during the culture period so that the oil concentration in the culture supernatant fell in the range of 0.3 to 2%.

In the main culture, a phosphoric acid solution was also intermittently added as a phosphorus source during the culture period.

The main culture medium used was a culture medium A shown in Table 1. At the start of the main culture, the sulfur concentration in the culture medium A containing the seed culture fluid was 16.0 mM.

The main culture was conducted for 72 hours.

TABLE 1

| | Molecular | Culture | Culture | Culture | Culture | Culture |
|---|---|---|---|---|---|---|
| Additives | weight | medium A | medium B | medium C | medium D | medium E |
| Na₂HPO₄•12H₂O (w/v %) | 163.94 | 0.385 | 0.385 | 0.385 | 0.385 | 0.385 |
| KH₂PO₄ (w/v %) | 136.09 | 0.067 | 0.067 | 0.067 | 0.067 | 0.067 |
| MgSO₄•7H₂O (w/v %) | 246.47 | 0.100 | 0.100 | 0.100 | 0.100 | 0.100 |
| (NH₄)₂SO₄ (w/v %) | 132.14 | 0.150 | 0.125 | 0.100 | 0.060 | 0.015 |
| NH₄Cl (w/v %) | 53.49 | 0.114 | 0.134 | 0.155 | 0.187 | 0.223 |
| Trace metal salt solution | — | 0.500 | 0.500 | 0.500 | 0.500 | 0.500 |
| (w/v %) (see Table 2) | | | | | | |
| Sulfur concentration (mM) | — | 15.4 | 13.5 | 11.6 | 8.6 | 5.2 |
| (Before addition of seed | | | | | | |
| culture fluid) | | | | | | |
| Sulfur concentration (mM) | | 16.0 | 14.1 | 12.2 | 9.2 | 5.8 |
| (Inclusive of sulfur source | | | | | | |
| of seed culture fluid) | | | | | | |

TABLE 2

| Trace metal salt solution | Molecular | |
|---|---|---|
| (0.1N hydrochloric acid) | weight | Concentration |
| FeCl₃•6H₂O (w/v %) | 270.29 | 1.600 |
| CaCl₂•2H₂O (w/v %) | 147.01 | 1.000 |
| CoCl₂•6H₂O (w/v %) | 237.93 | 0.020 |
| CuSO₄•5H₂O (w/v %) | 249.69 | 0.016 |
| NiCl₂•6H₂O (w/v %) | 129.60 | 0.012 |
| Sulfur concentration (mM) | — | 0.6 |

After the end of the culture, a certain volume of the culture fluid was extracted, and the microbial cells collected from the culture fluid were washed with distilled water and ethanol and then vacuum-dried. The weight of the PHA-containing dried microbial cells was measured. In addition, the microbial cells washed as described above were suspended in an aqueous solution of SDS, and the microbial cells in the suspension were subjected to ultrasonic disruption to disrupt and dissolve out cellular components and separate the PHA from the cellular components. Only the PHA was collected by centrifugation, and the amount of the accumulated PHA was measured. Based on the measurement result, the PHA productivity was calculated. The result of the PHA productivity calculation is shown in Table 3.

### Reference Example 2

(1) Preculture, (2) seed culture, and (3) main culture were sequentially performed using the same conditions as in Reference Example 1, except that the main culture medium used was a culture medium B shown in Table 1. At the start of the main culture, the sulfur concentration in the culture medium B containing the seed culture fluid was 14.1 mM.

After the end of the culture, the PHA productivity was calculated using the same conditions as in Reference Example 1. The result of the PHA productivity calculation is shown in Table 3.

### Comparative Example 1

(1) Preculture, (2) seed culture, and (3) main culture were sequentially performed using the same conditions as in Reference Example 1, except that the main culture medium used was a culture medium C shown in Table 1. At the start of the main culture, the sulfur concentration in the culture medium C containing the seed culture fluid was 12.2 mM.

After the end of the culture, the PHA productivity was calculated using the same conditions as in Reference Example 1. The result of the PHA productivity calculation is shown in Table 3.

### Comparative Example 2

(1) Preculture, (2) seed culture, and (3) main culture were sequentially performed using the same conditions as in Reference Example 1, except that the main culture medium used was a culture medium D shown in Table 1. At the start of the main culture, the sulfur concentration in the culture medium D containing the seed culture fluid was 9.2 mM.

After the end of the culture, the PHA productivity was calculated using the same conditions as in Reference Example 1. The result of the PHA productivity calculation is shown in Table 3.

### Comparative Example 3

(1) Preculture, (2) seed culture, and (3) main culture were sequentially performed using the same conditions as in Reference Example 1, except that the main culture medium used was a culture medium E shown in Table 1. At the start of the main culture, the sulfur concentration in the culture medium E containing the seed culture fluid was 5.8 mM.

After the end of the culture, the PHA productivity was calculated using the same conditions as in Reference Example 1. The result of the PHA productivity calculation is shown in Table 3.

### Examples 1, 3, 5, 7, 9, and 11

(1) Preculture, (2) seed culture, and (3) main culture were sequentially performed using the same conditions as in Comparative Example 1, except that in (3) main culture, a 43 g/L aqueous solution of sodium sulfate was added 20 hours after the start of the culture and thereafter was added intermittently until the end of the culture. The ratios (C/S ratios) of the carbon weight (C) in the carbon source added to the sulfur weight (S) in the sulfur source added were calculated for the period from the start of the addition of the aqueous solution of sodium sulfate to the end of the culture, and the average C/S ratio was calculated. The results are shown in Table 3.

For the period until the end of the addition (namely, the period from the 20th hour to the 72nd hour), the ratios (C/S ratio) of the carbon weight (C) in the carbon source added per hour to the sulfur weight (S) in the sulfur source added per hour were calculated. The minimum, maximum, and average of the C/S ratios are listed in Table 3.

The carbon weight (C) in the carbon source added per hour was calculated by the following equation.

(Weight (g) of palm olein oil added per hour)/(molecular weight of palm olein oil triglyceride)×(number of carbon atoms in molecule of palm olein oil triglyceride)×(molecular weight of carbon)

The molecular weight and carbon number of palm olein oil triglyceride were calculated by analyzing the fatty acid composition of palm olein oil and assuming that palm olein oil was composed of 100% triglyceride.

The sulfur weight (S) in the sulfur source added per hour was calculated by the following equation.

(Concentration (g/L) of aqueous sodium sulfate solution added)/(specific gravity of aqueous sodium sulfate solution at that concentration)×(weight of aqueous sodium sulfate solution added per hour)/(molecular weight of sodium sulfate)×(molecular weight of sulfur)

After the end of the culture, the PHA productivity was calculated using the same conditions as in Reference Example 1. The results of the PHA productivity calculation are shown in Table 3.

### Examples 2, 4, 6, 8, 10, and 12

(1) Preculture, (2) seed culture, and (3) main culture were sequentially performed using the same conditions as in Comparative Example 1, except that in (3) main culture, a 43 g/L aqueous solution of sodium sulfate was added 40 hours after the start of the culture and thereafter was added intermittently until the end of the culture. The minimum and maximum of the C/S ratios were determined using the same conditions as in Examples 1, 3, 5, 7, 9, and 11, and the average of the C/S ratios was also calculated. The minimum, maximum, and average of the C/S ratios are listed in Table 3.

After the end of the culture, the PHA productivity was calculated using the same conditions as in Reference Example 1. The results of the PHA productivity calculation are shown in Table 3.

TABLE 3

| | | Initial | | | | | PHA |
|---|---|---|---|---|---|---|---|
| | | Sulfur | | | | PHA | produc- |
| | | concen- | | | | produc- | tivity |
| | | tration | C/S ratio | | | tivity | (compared |
| | Way of supplying sulfur source | (mM) | Minimum | Average | Maximum | (g/L) | to Comp. 1) |
| Ref. 1 | Only initial addition | 16.0 | — | — | — | 185 | 81 |
| Ref. 2 | Only initial addition | 14.1 | — | — | — | 210 | 91 |
| Comp. 1 | Only initial addition | 12.2 | — | — | — | 233 | 100 |
| Comp. 2 | Only initial addition | 9.2 | — | — | — | 200 | 88 |
| Comp. 3 | Only initial addition | 5.8 | — | — | — | 143 | 64 |
| Ex. 1 | Initial addition + addition of Na | 12.2 | 5189 | 7213 | 9548 | 238 | 102 |
| | sulfate at and after 20th hour | | | | | | |
| Ex. 2 | Initial addition + addition of Na | 12.2 | 4826 | 7062 | 8687 | 242 | 104 |
| | sulfate at and after 40th hour | | | | | | |
| Ex. 3 | Initial addition + addition of Na | 12.2 | 2698 | 3887 | 5812 | 248 | 106 |
| | sulfate at and after 20th hour | | | | | | |
| Ex. 4 | Initial addition + addition of Na | 12.2 | 2595 | 3365 | 4567 | 249 | 107 |
| | sulfate at and after 40th hour | | | | | | |
| Ex. 5 | Initial addition + addition of Na | 12.2 | 2574 | 2684 | 3217 | 255 | 109 |
| | sulfate at and after 20th hour | | | | | | |
| Ex. 6 | Initial addition + addition of Na | 12.2 | 2059 | 2522 | 3539 | 251 | 107 |
| | sulfate at and after 40th hour | | | | | | |
| Ex. 7 | Initial addition + addition of Na | 12.2 | 1287 | 2623 | 3603 | 255 | 109 |
| | sulfate at and after 20th hour | | | | | | |
| Ex. 8 | Initial addition + addition of Na | 12.2 | 1287 | 2263 | 3217 | 254 | 109 |
| | sulfate at and after 40th hour | | | | | | |
| Ex. 9 | Initial addition + addition of Na | 12.2 | 1010 | 1557 | 2170 | 253 | 108 |
| | sulfate at and after 20th hour | | | | | | |
| Ex. 10 | Initial addition + addition of Na | 12.2 | 1010 | 1567 | 2245 | 254 | 109 |
| | sulfate at and after 40th hour | | | | | | |
| Ex. 11 | Initial addition + addition of Na | 12.2 | 643 | 1395 | 2091 | 253 | 108 |
| | sulfate at and after 20th hour | | | | | | |
| Ex. 12 | Initial addition + addition of Na | 12.2 | 374 | 1588 | 2245 | 250 | 107 |
| | sulfate at and after 40th hour | | | | | | |
| Ex. 13 | Initial addition + addition of K | 12.2 | 1908 | 2449 | 3307 | 253 | 108 |
| | sulfate at and after 30th hour | | | | | | |
| Ex. 14 | Initial addition +addition of Na | 9.2 | 647 | 1364 | 2363 | 253 | 109 |
| | sulfate at and after 10th hour | | | | | | |
| Ex. 15 | Initial addition + addition of Na | 5.8 | 748 | 1315 | 1871 | 251 | 107 |
| | sulfate at and after 10th hour | | | | | | |

Table 3 reveals the following facts. Comparative Examples 1 to 3 demonstrate that in the case where the sulfur concentration in the culture medium at the start of the culture is in a range up to 13 mM, the PHA productivity is enhanced with increasing sulfur concentration. However, in Reference Examples 1 and 2 where the sulfur concentration was higher than 13 mM, the PHA productivity was lower than in Comparative Example 1 where the sulfur concentration was 12.2 mM. This demonstrates that the PHA production is inhibited when the sulfur concentration at the start of the culture is higher than 13 mM.

In Examples 1 to 12, the sulfur concentration in the culture medium at the start of the culture was not higher than 13 mM, and a sulfur source was intermittently added during the culture so that the average C/S ratio fell within a given range. In consequence, the PHA productivity was higher than in Comparative Example 1 where no sulfur source was added during the culture. In, among others, Examples 3 to 12 where the average C/S ratio was not more than 6,000, the productivity was more than 5% higher than that in Comparative Example 1.

### Example 13

(1) Preculture, (2) seed culture, and (3) main culture were sequentially performed using the same conditions as in Comparative Example 1, except that in (3) main culture, a 31 g/L aqueous solution of potassium sulfate was added 30 hours after the start of the culture and thereafter was added intermittently until the end of the culture. The minimum and maximum of the C/S ratios were determined using the same conditions as in Examples 1, 3, 5, 7, 9, and 11, and the average of the C/S ratios was also calculated. The minimum, maximum, and average of the C/S ratios are listed in Table 3.

The sulfur weight (S) in the sulfur source added per hour was calculated by the following equation.

(Concentration (g/L) of aqueous potassium sulfate solution added)/(specific gravity of aqueous potassium sulfate solution at that concentration)×(weight of aqueous potassium sulfate solution added per hour)/(molecular weight of potassium sulfate)×(molecular weight of sulfur)

After the end of the culture, the PHA productivity was calculated using the same conditions as in Reference Example 1. The result of the PHA productivity calculation is shown in Table 3. In Example 13, the PHA productivity was more than 5% higher than that in Comparative Example 1.

### Example 14

(1) Preculture, (2) seed culture, and (3) main culture were sequentially performed using the same conditions as in Comparative Example 2, except that in (3) main culture, a 43 g/L aqueous solution of sodium sulfate was added 10 hours after the start of the culture and thereafter was added intermittently until the end of the culture. The minimum and maximum of the C/S ratios were determined using the same conditions as in Examples 1, 3, 5, 7, 9, and 11, and the average of the C/S ratios was also calculated. The minimum, maximum, and average of the C/S ratios are listed in Table 3.

After the end of the culture, the PHA productivity was calculated using the same conditions as in Reference Example 1. The result of the PHA productivity calculation is shown in Table 3. In Example 14, the PHA productivity was more than 20% higher than that in Comparative Example 2 where the sulfur concentration at the start of the main culture was the same as in Example 14. The PHA productivity in Example 14 was more than 5% higher than that in Comparative Example 1.

### Example 15

(1) Preculture, (2) seed culture, and (3) main culture were sequentially performed using the same conditions as in Comparative Example 3, except that in (3) main culture, a 43 g/L aqueous solution of sodium sulfate was added 10 hours after the start of the culture and thereafter was added intermittently until the end of the culture. The minimum and maximum of the C/S ratios were determined using the same conditions as in Examples 1, 3, 5, 7, 9, and 11, and the average of the C/S ratios was also calculated. The minimum, maximum, and average of the C/S ratios are listed in Table 3.

After the end of the culture, the PHA productivity was calculated using the same conditions as in Reference Example 1. The result of the PHA productivity calculation is shown in Table 3. In Example 15, the PHA productivity was more than 60% higher than that in Comparative Example 3 where the sulfur concentration at the start of the main culture was the same as in Example 15. The PHA productivity in Example 15 was more than 5% higher than that in Comparative Example 1.

1: A polyhydroxyalkanoate production method comprising:
culturing a polyhydroxyalkanoate-producing microorganism in a culture medium to obtain microbial cells accumulating a polyhydroxyalkanoate,
wherein at start of the culturing of the polyhydroxyalkanoate-producing microorganism, the culture medium comprises a sulfur source at a sulfur concentration of from 0.0001 to 13 mM, and
adding a carbon source and a sulfur source to the culture medium during the culturing of the polyhydroxyalkanoate-producing microorganism,
wherein an average ratio (C/S ratio) of a carbon weight (C) in the carbon source added per hour to a sulfur weight (S) in the sulfur source added per hour, as calculated for a period in which the sulfur source is added, is from 500 to 10,000.

2: The polyhydroxyalkanoate production method according to claim 1, wherein the average C/S ratio is from 1,000 to 6,000.

3: The polyhydroxyalkanoate production method according to claim 1, wherein the sulfur source comprises at least one substance selected from the group consisting of sulfuric acid and a sulfate salt.

4: The polyhydroxyalkanoate production method according to claim 1, wherein the polyhydroxyalkanoate-producing microorganism belongs to the genus Cupriavidus.

5: The polyhydroxyalkanoate production method according to claim 1, wherein the polyhydroxyalkanoate-producing microorganism is Cupriavidus necator.

6: The polyhydroxyalkanoate production method according to claim 1, wherein the polyhydroxyalkanoate is a copolymer comprising at least 3-hydroxybutyrate and 3-hydroxyhexanoate as monomer units.
